(19) United States
(12) Patent Application Publication
(10) Pub. No.: US 2024/0409904 A1
(43) Pub. Date: Dec. 12, 2024

(54) PROTOCOL GENERATING TISSUE SPECIFIC ENDOTHELIAL CELL LINE, NAMED LIOVECS (LUNG TISSUE IPSC ORIGIN VESSEL ORGANOID ENDOTHELIAL CELL) AND BIOVECS (BLOOD VESSEL STEM CELL IPSC ORIGIN VESSEL ORGANOID ENDOTHELIAL CELLS)

(71) Applicant: THE CATHOLIC UNIVERSITY OF KOREA INDUSTRY-ACADEMIC COOPERATION FOUNDATION, Seoul (KR)

(72) Inventors: Sung Won KIM, Seoul (KR); Ayoung JO, Seoul (KR); Min Jae LIM, Gyeonggi-do (KR)

(21) Appl. No.: 18/737,066

(22) Filed: Jun. 7, 2024

(30) Foreign Application Priority Data
Jun. 7, 2023 (KR) 10-2023-0073162

(51) Int. Cl. C12N 5/071

(52) U.S. Cl. CPC C12N 5/0691; C12N 2506/1369; C12N 2509/00; C12N 2513/00

(57) ABSTRACT

The present invention relates to a method of developing a tissue-specific vascular endothelial cell line based on iPSCs derived from human tissue-specific cells. When an organoid is manufactured using the cell line based on the iPSCs derived from human tissue-specific cells, the organoid manufacturing period may be significantly shortened, thereby solving the problem of sustainability, which is a major drawback known in the related art, and may be stored conveniently, and thus may be easily used during each experiment, thereby greatly increasing efficiency. Also, the organoid manufactured from the cell line of the present invention may be usefully used in the manufacture of blood vessel organoids in that the organoid is not only tissue-specific but also exhibits an excellent level of morphological and functional maturity.

### CROSS-REFERENCE TO RELATED APPLICATIONS

This application claims priority to Korean Patent Application No. 10-2023-0073162, filed on Jun. 7, 2023, the disclosure of which is incorporated herein by reference in its entirety.

### TECHNICAL FIELD

The present invention relates to a method of developing a tissue-specific vascular endothelial cell line based on iPSCs derived from human tissue-specific cells.

### BACKGROUND ART

In the case of primary culture cell models currently used in laboratories, cells derived from organs or tissues are artificially immortalizing or two-dimensionally cultured to verify the efficacy and toxicity of candidate substances. However, the cells artificially cultured on 2D have the fatal flaw of failing to reflect actual phenomena occurring in living organisms because changes in original cell shape and loss of cell function occur during the culture process after separation from tissue, or the cells are disconnected with the extracellular environment (ECM; a microenvironment). In addition, in the case of non-human cells, the action and mechanism of a drug are often not the same as in humans due to differences between species. Therefore, the non-human cells have several problems in that the development of efficient drugs is hindered and a lot of time and money is required, which makes it difficult to screen drugs and predict reactivity.

In order to solve these problems, the development of stem cell organoid manufacturing technology has received recent attention as a method of manufacturing organoids and utilizing the organoids for disease modeling, pathology research, drug screening, toxicity evaluation, and genetic manipulation. When differentiation into a 3D state was induced using the multipotency and intrinsic self-organization of stem cells, it was confirmed that a 3D organoid construct having a structure similar to a body organ was formed. To date, results have been reported that various organoids, including brain organoids, heart organoids, liver organoids, lung organoids, blood vessel organoids, and the like, can be manufactured based on the above finding.

These organoids contain several specific cell populations that make up an organ or tissue, have a morphological and structural organization similar to actual tissues or organs, and may reproduce the special functions of each organ. Organoids are formed through a series of common processes. Cells with the same function are grouped together and placed in an appropriate location. After the cell compartments are separated, more sophisticated differentiation may occur. This is called lineage specification, which is a process of differentiating precursor cells into complete adult cells so that they can perform actual functions.

Also, cell lines having excellent functionality are prepared, stored, and used for various types of research. A cell line refers to cultured cells having the same characteristics, and cell lines from various tissues have been used for this purpose. A representative example is a vascular endothelial cell line.

Because the development of these cell lines or organoids creates a 3D environment close to the inside of the living body, allowing experiments to be conducted “outside the body” as if the drug is acting “inside the body” even in an experimental situation. Therefore, such cell lines or organoids have the advantage of replicating the effects that appear in actual human organs, which makes it possible to perform new drug screening, drug toxicity testing, and the like.

Despite these numerous advantages and necessities, there is no report on a method of manufacturing a vascular endothelial cell line or a blood vessel organoid with high functionality.

### RELATED-ART DOCUMENT

### Patent Document

Korean Patent Publication No. 10-2016-0121240

### DISCLOSURE

### Technical Problem

Accordingly, it is an object of the present invention to provide a vascular endothelial cell line derived from induced pluripotent stem cells (iPSCs).

It is another object of the present invention to provide a method of producing a vascular endothelial cell line, comprising:
manufacturing a blood vessel organoid from induced pluripotent stem cells; and
dissociating and culturing single vascular endothelial cells from the blood vessel organoid.

It is still another object of the present invention to provide a method of manufacturing a blood vessel organoid from a vascular endothelial cell line, comprising: culturing the cell line in a maturation medium.

It is yet another object of the present invention to provide a blood vessel organoid manufactured by the method.

It is yet another object of the present invention to provide a kit for manufacturing a blood vessel organoid, comprising the cell line and instructions.

It is yet another object of the present invention to provide a kit for preparing a vascular endothelial cell line, comprising induced pluripotent stem cells and instructions.

However, the technical objects to be achieved by the present invention are not limited to the above-described technical objects, and other objects which are not mentioned above will be clearly understood from the following detailed description by those skilled in the art to which the present invention pertains.

### Technical Solution

According to an aspect of the present invention, there is provided a vascular endothelial cell line derived from induced pluripotent stem cells (iPSCs).

According to one embodiment of the present invention, the induced pluripotent stem cells may be derived from lung cells or umbilical cord blood, but the present invention is not limited thereto.

According to one embodiment of the present invention, the cell line may be prepared from the blood vessel organoid derived from the induced pluripotent stem cells, but the present invention is not limited thereto.

According to one embodiment of the present invention, the cell line may express any one or more vascular differentiation markers selected from the group consisting of CD144, CD146, CD31, and CD140b, but the present invention is not limited thereto.

According to one embodiment of the present invention, the cell line may be used to manufacture a blood vessel organoid, but the present invention is not limited thereto.

According to another aspect of the present invention, there is provided a method of producing a vascular endothelial cell line, comprising:
manufacturing a blood vessel organoid from induced pluripotent stem cells; and
dissociating and culturing single vascular endothelial cells from the blood vessel organoid.

According to one embodiment of the present invention, the culturing may be a process of culturing the cells in an endothelial growth medium (EGM), but the present invention is not limited thereto.

According to one embodiment of the present invention, the induced pluripotent stem cells may be derived from lung cells or umbilical cord blood, but the present invention is not limited thereto.

According to still another aspect of the present invention, there is provided a method of manufacturing a blood vessel organoid from a vascular endothelial cell line, comprising: culturing the cell line in a maturation medium to manufacture the blood vessel organoid.

According to one embodiment of the present invention, the method may not include any one or more of the following steps, but the present invention is not limited thereto:
culturing induced pluripotent stem cells in an aggregate-forming medium;
culturing the cells in a mesodermal induction medium; and
culturing the cells in a vascular induction medium.

According to yet another aspect of the present invention, there is provided a blood vessel organoid manufactured by the method.

According to one embodiment of the present invention, the organoid may express any one or more blood vessel-specific proteins selected from CD31 or αSMA, but the present invention is not limited thereto.

According to yet another aspect of the present invention, there is provided a kit for manufacturing a blood vessel organoid, comprising the cell line and instructions.

According to yet another aspect of the present invention, there is provided a kit for preparing a vascular endothelial cell line, comprising the induced pluripotent stem cells and instructions.

According to yet another aspect of the present invention, there is provided a use of the induced pluripotent stem cells (iPSCs) or the blood vessel organoid derived from the induced pluripotent stem cells for the production of a vascular endothelial cell line.

According to yet another aspect of the present invention, there is provided a use of the induced pluripotent stem cells (iPSCs) or the vascular endothelial cell line derived from the induced pluripotent stem cells for the manufacture of a blood vessel organoid.

### Advantageous Effects

In the progress of research on blood vessel organoids that can enable the production of blood vessels to increase the similarity of organoids to the actual human body and the direct provision of blood vessels, tissue-specific blood vessel organoids are formed from iPSCs for specific tissues, and vascular cell lines derived from tissue-specific blood vessel organoids are produced to increase the convenience of the one-time experiment method of blood vessel organoids produced from iPSCs that are currently already in use, thereby providing convenience during research and increasing actual differentiation efficiency as well.

Also, the organoids manufactured from the cell line of the present invention can be usefully used in the manufacture of blood vessel organoids in that they are not only tissue-specific but also exhibit an excellent level of morphological and functional maturity.

### DESCRIPTION OF DRAWINGS

FIG. 1A is a schematic diagram showing a protocol for manufacturing a blood vessel organoid from iPSCs.

FIG. 1B is an image showing the shapes of organoids at each time point in FIG. 1A.

FIG. 1C is an image showing the results of a staining experiment confirming the maturation levels of blood vessel organoids manufactured from iPSCs.

FIG. 2 is a schematic diagram and image showing a process of producing a vascular endothelial cell line of the present invention.

FIG. 3 is a graph showing the results of an experiment comparing the expression levels of specific factors related to vascular differentiation when LIOVECs and BIOVECs are compared with HUVECs.

FIG. 4 is an image showing a process of forming a network of blood vessels during angiogenic differentiation using LIOVECs and BIOVECs.

### BEST MODE

The present invention provides a vascular endothelial cell line derived from induced pluripotent stem cells (iPSCs). Preferably, the present invention provides a vascular endothelial cell line derived from blood vessel organoids manufactured from induced pluripotent stem cells.

In the present invention, the term “cell line” refers to a clone of cultured cells that may continue to divide and proliferate through cell culture and pass on genetic characteristics to subsequent generations, that is, a series of cell lines that maintain their genetic characteristics even when subcultured. Therefore, the vascular endothelial cell line of the present invention may refer to a collection of cultured cells that may divide and proliferate through cell culture and pass on genetic characteristics to subsequent generations while maintaining the characteristics of vascular endothelial cells. In particular, the vascular endothelial cell line of the present invention is produced from blood vessel organoids manufactured from iPSCs. In this case, because the organoid significantly expresses blood vessel-specific markers and has a high level of maturity, the vascular endothelial cell line produced from the organoid also has significantly superior vascular differentiation efficiency, which makes it possible to functionally preserve vascular endothelial cells as is. Therefore, the vascular endothelial cell line may be used for various research purposes associated with vascular endothelial cells.

In the present invention, the term “induced pluripotent stem cells (iPSCs),” which are also referred to as reprogrammed stem cells, are somatic cells or cells, which have completed differentiation, that are reset (reprogrammed) to a pluripotent embryonic stem cell-like state through a specific method. In this case, methods of inducing iPSCs includes methods such as genetic transformation by injection of cell differentiation-related genes, culturing under specific conditions, and the like, but the present invention is not limited thereto.

Induced pluripotent stem cells may be produced from any somatic cells or any cells that have completed differentiation. In the present invention, the induced pluripotent stem cells may be prepared from peripheral blood mononuclear cells, but the present invention is not limited thereto.

In the present invention, the term “organoid” refers to an organ-specific cell aggregate made by three-dimensionally culturing, aggregating or recombining stem cells, is capable of self-renewal, and is also called “mini-organ” or “pseudo-organ.” Organoids have a shape similar to actual organs and may be used to in vitro carry out studies that are difficult to implement in animal models, such as molecular signaling regulation. Therefore, the organoids are very useful in basic research and may be very usefully used in various fields such as human developmental processes, disease model establishment, screening for drug effectiveness evaluation, development of drug toxicity evaluation platform, cell therapy development, and the like, but the present invention is not limited thereto.

According to one embodiment of the present invention, the induced pluripotent stem cells may be derived from lung cells or umbilical cord blood, but the present invention is not limited thereto.

In the present invention, the term “lung cells” may be “alveolar epithelium,” preferably “alveolar type 1 cells” or “alveolar type 2 cells,” and more preferably “alveolar type 2 cells,” but the present invention is not limited thereto.

The alveolar epithelium is not only essential for the gas exchange function in the lungs, but also serves as an important barrier protecting the body from danger. In response to acute injury, the alveoli can rapidly repair and regenerate new alveolar epithelial cells to restore an intact epithelial barrier. The alveolar epithelium is mainly composed of two types of epithelial cells: alveolar type I (AT1) and type II (AT2) cells. AT2 cells are smaller cuboidal cells best known for their ability to synthesize and secrete a pulmonary surfactant, serve as alveolar stem cells, and can differentiate into AT1 cells during alveolar homeostasis and repair after injury. In general, type 1 alveolar cells constitute the main gas exchange surface of the alveoli and perform an integral function in maintaining the permeability barrier function of the alveolar membrane, while type 2 alveolar cells are the precursors of type 1 cells and may be responsible for surfactant production and homeostasis.

In the present invention, the term “umbilical cord blood” is defined as blood collected from the umbilical vein connecting the fetus and the placenta. Umbilical cord blood is a by-product that naturally occurs during childbirth, and is much easier to collect than general mesenchymal tissue such as bone marrow, which requires multiple surgeries. Compared to bone marrow transplantation, the cord blood storage industry has become active and its infrastructure has already been established, which makes it easy to find donors. In addition, because umbilical cord blood-derived cells do not express the histocompatibility antigen HLA-DR (class II), which is the most important cause of rejection in tissue or organ transplantation (Le Blanc, K C, Exp Hematol, 31:890-896, 2003; and Tse W T et al., Transplantation, 75:389-397, 2003), the umbilical cord blood-derived cells may not induce or may minimize immune responses such as rejection, which were problematic during conventional transplant surgery. As a result, both autologous umbilical cord blood as well as allogeneic umbilical cord blood can be used, but the present invention is not limited thereto.

In the present invention, when the AT2 cell- or umbilical cord blood-derived induced pluripotent stem cells are used, it is possible to manufacture blood vessel organoids that not only preserve the function of vascular endothelial cells but also express large amounts of vascular differentiation markers. Also, a vascular cell line having significantly higher vascular differentiation efficiency may be produced from the blood vessel organoids.

According to one embodiment of the present invention, the cell line may be produced from a blood vessel organoid derived from induced pluripotent stem cells, but the present invention is not limited thereto.

According to one embodiment of the present invention, the cell line may express any one or more vascular differentiation markers selected from the group consisting of CD144, CD146, CD31, and CD140b, but the present invention is not limited thereto.

In the present invention, the term “vascular differentiation marker” refers to a specific factor that appears when a vascular cell line differentiates into blood vessels, and includes all types of markers used to identify that a specific cell or tissue has differentiated into a vascular cell or vascular tissue when large amounts of the vascular differentiation markers are expressed. In this case, when a vascular differentiation marker is expressed in a specific cell or tissue, it may be judged to exhibit vascular tissue-specific characteristics, but the present invention is not limited thereto.

The vascular cell line of the present invention may be vascular tissue-specific by expressing large amounts of vascular differentiation markers. In the present invention, the term “vascular tissue-specific” means that cells exhibit characteristics generally displayed in vascular tissue, for example, morphological characteristics of vascular tissue. In addition, it may mean that proteins, mechanisms, and the like that are specifically found in vascular tissue appear so that a cell line can exhibit characteristics expected to perform the function of vascular tissue, but the present invention is not limited thereto.

In the present invention, the cell line may be characterized by having significantly high vascular differentiation efficiency because at least 95% of the cells express vascular differentiation markers based on the total number of cells.

In the vascular cell line of the present invention, most cells were observed to simultaneously express three or more vascular differentiation markers consisting of CD144, CD146, CD31, and CD140b. Therefore, because the functionality of vascular endothelial cells is preserved at a high level, the vascular cell line may be usefully used as a cell line for various studies such as screening, and the like. Also, the cell line of the present invention may be easily used because it is stored under specific conditions like cell banks, but the present invention is not limited thereto.

According to one embodiment of the present invention, the cell line may be used to manufacture blood vessel organoids, but the present invention is not limited thereto.

The cell line of the present invention is used to manufacture blood vessel organoids, and a kit commonly used in the art to manufacture organoids may be used. For example, the kit may be a STEMdiff blood vessel organoid kit, but the present invention is not limited thereto.

When the cell line according to the present invention is used, the minimum maturation period of 19 days (Stage 5 in the present invention) that must be cultured in a maturation medium may be significantly shortened, thereby reducing the time and costs and increasing the simplicity of procedures generally required to manufacture organoids, but the present invention is not limited thereto.

The present invention provides a method of producing a vascular endothelial cell line, comprising:
manufacturing a blood vessel organoid from induced pluripotent stem cells; and
dissociating and culturing single vascular endothelial cells from the blood vessel organoid.

In the present invention, the process of producing a tissue-specific vascular cell line from the completed blood vessel organoid includes the dissociation and culturing step, and may further include additional steps to produce a tissue-specific vascular cell line, but the present invention is not limited thereto.

According to one embodiment of the present invention, the culturing may be a process of culturing the cells in an endothelial growth medium (EGM), but the present invention is not limited thereto.

In the present invention, the endothelial growth medium may be one commonly used in the art, but the present invention is not limited thereto.

According to one embodiment of the present invention, the induced pluripotent stem cells may be derived from lung cells or umbilical cord blood, but the present invention is not limited thereto.

The present invention provides a method of manufacturing a blood vessel organoid from a vascular endothelial cell line, comprising: culturing the cell line in a maturation medium to manufacture a blood vessel organoid.

In the present invention, the term “maturation medium” refers to a medium used in a process of manufacturing blood vessel organoids. In the present invention, the maturation medium may be a STEMdiff™ Blood Vessel Organoid Maturation medium* (100-0658), but the present invention is not limited thereto.

According to one embodiment of the present invention, the method may be characterized by not including any one or more of the following steps, but the present invention is not limited thereto:
culturing induced pluripotent stem cells in an aggregate-forming medium;
culturing the cells in a mesodermal induction medium; and
culturing the cells in a vascular induction medium.

The present invention provides a blood vessel organoid manufactured by the method.

According to one embodiment of the present invention, the organoid may express any one or more blood vessel-specific proteins selected from CD31 or αSMA, but the present invention is not limited thereto.

The present invention provides a kit for manufacturing a blood vessel organoid, comprising the above cell line and instructions.

In the present invention, the kit may not include any one of an aggregate-forming medium, a mesodermal induction medium, and a vascular induction medium, but the present invention is not limited thereto.

In addition to the above components, the kit of the present invention may include other components, devices, materials, and the like commonly required in a method of improving the efficiency of a composition of the present invention to manufacture blood vessel organoids or storing or managing the composition of the present invention. Also, all components included in the kit may be used one or more times without any limitation, and there is no restriction on the order in which the respective substances are used. In this case, the application of each substance may be carried out simultaneously or at any time point.

Further, the present invention provides a kit for producing a vascular endothelial cell line, comprising the induced pluripotent stem cells and instructions.

In the present invention, the kit may further include a component configured to manufacture a blood vessel organoid from induced pluripotent stem cells, and other components such as an agent for dissociating a single cell from the blood vessel organoid, an agent for culturing the single vascular endothelial cell to obtain a vascular endothelial cell line, and the like, but the present invention is not limited thereto.

In addition to the above components, the kit of the present invention may include a component configured to improve the yield or culture rate of a vascular endothelial cell line of the composition of the present invention, a component configured to remove impurities from vascular endothelial cells, a component configured to increase the efficiency for the dissociation of vascular endothelial cells, or components that appear during the process of manufacturing a vascular endothelial cell line, for example, other components, devices, materials, and the like commonly required in a method of storing or managing induced pluripotent stem cells, blood vessel organoids, single vascular endothelial cells dissociated from the blood vessel organoids, and the like. Also, all components included in the kit may be used one or more times without any limitation, and there is no restriction on the order in which the respective substances are used. In this case, the application of each substance may be carried out simultaneously or at any time point.

The kit of the present invention may include a container in addition to the above agents and instructions. The container may serve to package the components and may also serve to store and secure the components. The material of the container may take the form of, for example, a bottle, a tub, a sachet, an envelope, a tube, an ampoule, and the like, which may be partially or entirely formed of plastic, glass, paper, foil, wax, and the like. The container may be equipped with a completely or partially removable cover that may initially be part of the container or may be attached to the container by mechanical, adhesive, or other means and may also be equipped with a stopper that allows access to the contents by a syringe needle. The kit may include an external package, and the external package may include instructions for use of the components, but the present invention is not limited thereto.

In the present invention, the term “screening” may mean that any substance having a specific target property is selected from a candidate group consisting of various substances using a specific manipulation or evaluation method.

### MODE FOR INVENTION

Hereinafter, preferred examples of the present invention are presented in order to aid in understanding the present invention. However, it should be understood that the following examples are provided only to make the present invention easier to understand and are not intended to limit the present invention.

### EXAMPLES

### Example 1: Manufacture of Organoids from iPSCs and Confirmation of Excellent Maturity of Organoids

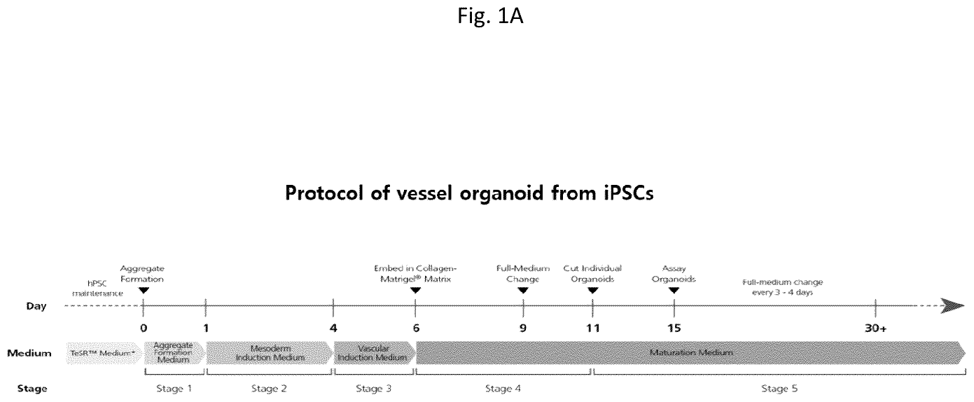

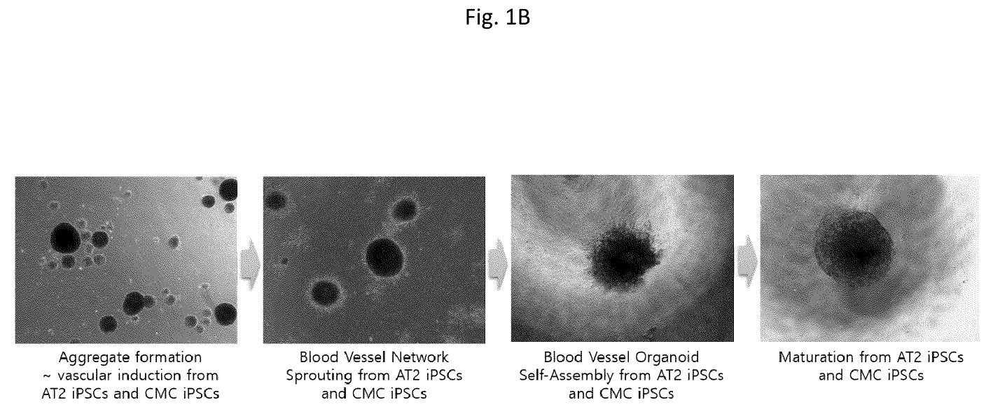

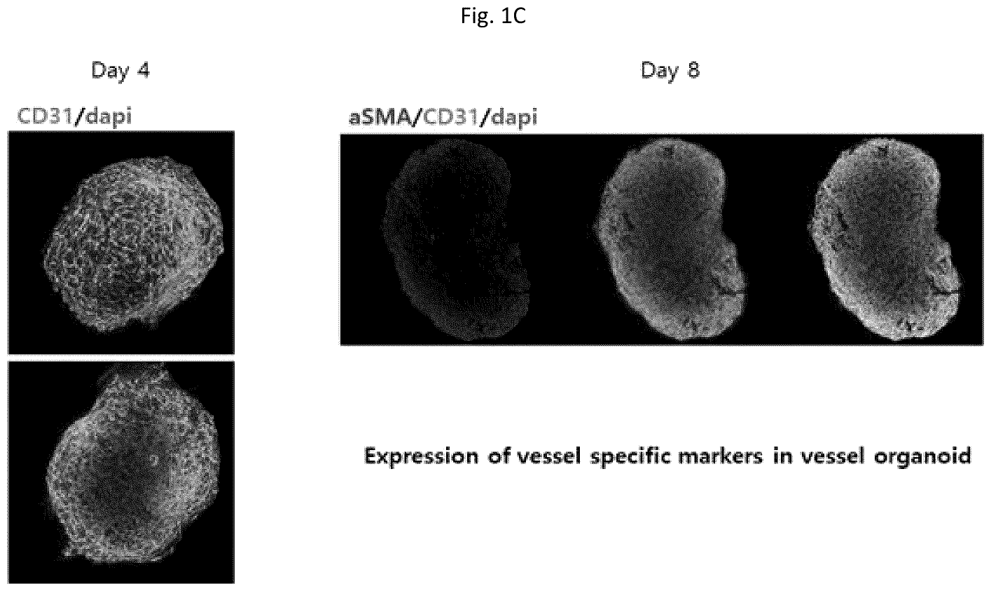

To produce the vascular endothelial cell line of the present invention, a blood vessel organoid was first manufactured from iPSCs. Specifically, a blood vessel organoid was manufactured using the STEMdiff blood vessel organoid kit and the method described in the instructions thereof. As a type of iPSCs, AT2 cells (alveolar type (AT) 2 pneumocytes), which belong to lung cells, were used to manufacture a blood vessel organoid (FIG. 1A).

As a result, as shown in FIG. 1B, it was morphologically confirmed that aggregates were formed from AT2 iPSCs and CMC iPSCs, and blood vessels were induced therefrom, and after a network of blood vessels was formed from the induced blood vessels, blood vessel organoids were not only self-assembled but also reached maturity.

Also, a functionally mature blood vessel organoid was manufactured in that the blood vessel-specific markers CD31 and αSMA of the organoid were expressed at significantly high levels (FIG. 1C).

### Example 2: Production of Tissue-Specific Vascular Cell Line

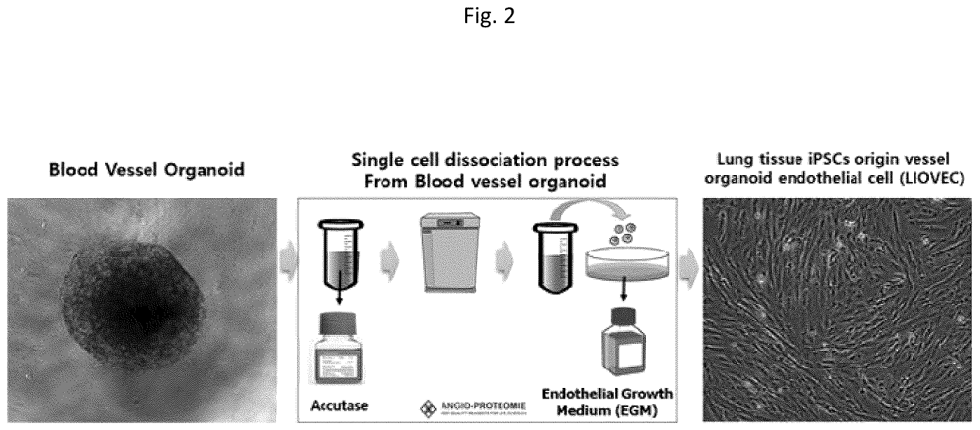

To produce a tissue-specific vascular cell line, the vascular organoid manufactured in Example 1 was separated into single cells. Specifically, the blood vessel organoid manufactured using iPSCs was treated with Accutase and cultured in a 37° C. incubator until the blood vessel organoid was separated into single cells. The separated single vascular endothelial cells were subcultured in a cell culture dish and used (FIG. 2).

As a result, it was confirmed that a tissue-specific vascular cell line was successfully produced. Based on these results, cells isolated from the blood vessel organoid manufactured using the type 2 alveolar cell-derived iPSCs were named lung tissue iPSC origin vessel organoid endothelial cells (LIOVECs), and cells isolated from the blood vessel organoid manufactured using the umbilical cord blood cell-derived iPSCs were named blood stem cell iPSC origin vessel organoid endothelial cells (BIOVECs).

### Example 3: Confirmation of Excellent Vascular Tissue Specificity of LIOVECs and BIOVECs

To confirm the vascular tissue specificity of LIOVECs and BIOVECs produced in Example 2, the expression levels of specific factors that appear during differentiation into blood vessels were analyzed. Specifically, the expression levels of vascular endothelial cell-specific factors CD144, CD146, CD31, and CD140b were checked through FACS, and a commonly used vascular endothelial cell line HUVEC was used as the control.

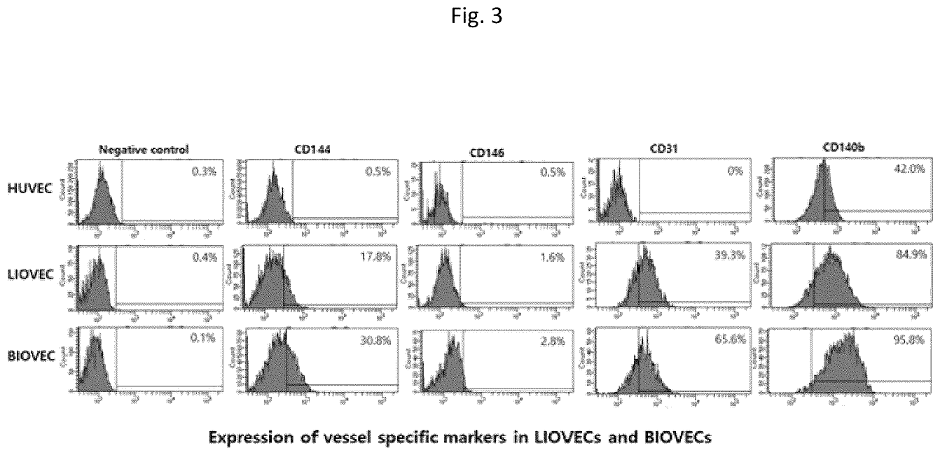

As a result, it was confirmed that the expression of the vascular tissue-specific factors was found to be significantly higher in LIOVECs and BIOVECs compared to HUVECs, and the vascular tissue specificity of the vascular cell line prepared in the present invention was superior to that of the previously used vascular cell line HUVEC (FIG. 3).

### Example 4: Confirmation of Excellent Vascular Differentiation Potential of LIOVECs and BIOVECs

The vascular differentiation potentials of LIOVECs and BIOVECs produced in Example 2 were analyzed. For this purpose, a process of forming a network of blood vessels was observed when differentiation for blood vessel formation proceeded with the cell line of the present invention. Specifically, LIOVECs or BIOVECs were mixed and cultured in a hydrogel containing a mixture of collagen, Matrigel and fibrin. For the differentiation potential into blood vessels, when tubes of blood vessels are formed, the thickness and the degree of connection between the formed tubes are very important factors in a morphological aspect. Therefore, an F-actin protein, which is able to recognize and stain human blood vessels from arteries to veins at once, was stained and then observed under a microscope to check whether the network of blood vessels was well formed based on the size of the tubes and the connectivity between blood vessels.

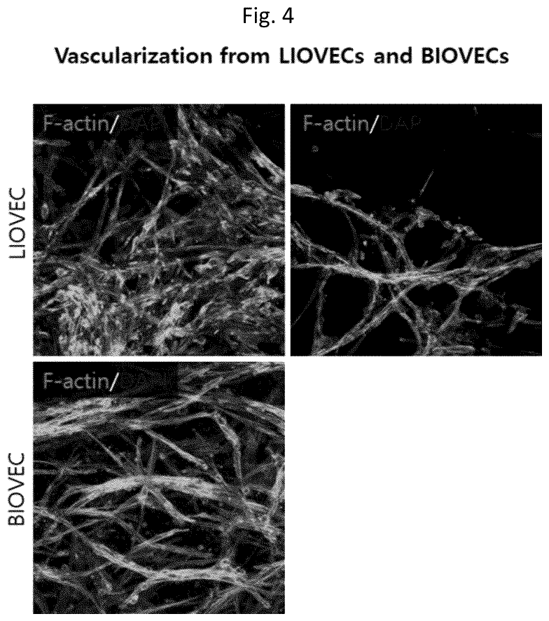

As a result, it was confirmed that LIOVECs and BIOVECs had excellent differentiation potentials, and the network of blood vessels was well formed when differentiation for blood vessel formation proceeded (FIG. 4).

Based on these results, it was confirmed that the cell line of the present invention was more efficient because the cell line was not only able to be conveniently used in each experiment, but also LIOVECs maintained in their original cell state were able to be continuously passaged, stored at −80° C., and used as cells like HUVECs.

The above description of the present invention is for illustrative purposes. Therefore, those skilled in the art to which the present invention pertains will appreciate that the present invention can be embodied in other specific forms without changing the technical spirit or essential features of the present invention. Therefore, it should be understood that the embodiments described above are illustrative in all respects and not restrictive.

1. A vascular endothelial cell line derived from induced pluripotent stem cells (iPSCs).

2. The vascular endothelial cell line of claim 1, wherein the induced pluripotent stem cells are derived from lung cells or umbilical cord blood.

3. The vascular endothelial cell line of claim 1, wherein the vascular endothelial cell line is produced from induced pluripotent stem cell-derived blood vessel organoids.

4. The vascular endothelial cell line of claim 1, wherein the vascular endothelial cell line expresses any one or more vascular differentiation markers selected from the group consisting of CD144, CD146, CD31, and CD140b.

5. The vascular endothelial cell line of claim 1, wherein the vascular endothelial cell line is used to manufacture blood vessel organoids.

6. A method of producing a vascular endothelial cell line, comprising:
manufacturing a blood vessel organoid from induced pluripotent stem cells; and
dissociating and culturing single vascular endothelial cells from the blood vessel organoid.

7. The method of claim 6, wherein the culturing is a process of culturing the single vascular endothelial cells in an endothelial growth medium (EGM).

8. The method of claim 6, wherein the induced pluripotent stem cells are derived from lung cells or umbilical cord blood.

9. A method of manufacturing a blood vessel organoid from a vascular endothelial cell line, comprising:
culturing the cell line of claim 1 in a maturation medium to manufacture the blood vessel organoid.

10. The method of claim 9, wherein the method does not include any one or more of the following steps:
culturing induced pluripotent stem cells in an aggregate-forming medium;
culturing the cells in a mesodermal induction medium; and
culturing the cells in a vascular induction medium.

11. The method of claim 9, wherein the blood vessel organoid expresses any one or more blood vessel-specific proteins selected from CD31 or αSMA.
